(19) United States
(12) Patent Application Publication
(10) Pub. No.: US 2020/0000961 A1
(43) Pub. Date: Jan. 2, 2020

(54) METHOD OF MANUFACTURING COMPOSITE MATERIAL SHAPED ARTICLE CONTAINING ACICULAR HYDROXYAPATITE, AND COMPOSITE MATERIAL SHAPED ARTICLE

(71) Applicant: SINTOKOGIO, LTD., Nagoya-shi, Aichi (JP)

(72) Inventors: Norihiro ASANO, Toyokawa-shi, Aichi (JP); Chikara OHTSUKI, Nagoya-shi, Aichi (JP)

(73) Assignee: SINTOKOGIO, LTD., Nagoya-shi, Aichi (JP)

(21) Appl. No.: 16/484,282

(22) PCT Filed: Feb. 21, 2018
(86) PCT No.: PCT/JP2018/006269

§ 371 Date: Aug. 7, 2019

(30) Foreign Application Priority Data
Feb. 28, 2017 (JP) 2017-036937

(51) Int. Cl. A61L 27/12; A61L 27/42; A61L 27/20; C04B 35/447

(52) U.S. Cl. CPC A61L 27/12; A61L 27/425; A61L 27/20; C04B 2235/606; A61L 2400/12; C04B 2235/5212; C04B 35/447

(57) ABSTRACT

A manufacturing method is a method of manufacturing a composite material molded article containing acicular hydroxyapatite. This manufacturing method comprises: a preparation step of mixing at least a calcium phosphate compound including α-tricalcium phosphate, a calcium compound containing no phosphorus, cellulose nanofibers, and an aqueous solvent consisting of water and/or a hydrophilic solvent to obtain a mixture; a molding step of forming a molded article by using the mixture; a drying step of drying the molded article; and a synthesis step of performing synthesis treatment of the molded article after drying.

### TECHNICAL FIELD

The present disclosure relates to a method of manufacturing a composite material molded article containing acicular hydroxyapatite, and the composite material molded article.

### BACKGROUND ART

Calcium phosphate particles formed in an acicular shape are a useful material as a biomaterial, a filling material for columns, and a filler for reinforcing composite materials. Especially, fine hydroxyapatite formed in a highly acicular shape can be a material exhibiting biological tissue affinity for living bone and a specific protein adsorption property.

Methods involving mixing a raw material containing a calcium compound and a phosphorus compound or a raw material containing calcium phosphate with water or a hydrophilic organic solvent and performing hydrothermal synthesis at 120° C. or more under a pressurized condition are known as methods of producing acicular hydroxyapatite (for example, refer to Patent Literatures 1 and 2).

### CITATION LIST

### Patent Literature

Patent Literature 1: Japanese Unexamined Patent Application Publication No. 2001-287903

Patent Literature 2: Japanese Unexamined Patent Application Publication No. 2002-274822

### SUMMARY OF INVENTION

### Technical Problem

However, when the acicular hydroxyapatite particles obtained by the methods disclosed in Patent Literatures 1 and 2 are used for a material replacing living hard tissue such as artificial bone or artificial teeth or an alternative material to ivory that is difficultly obtained, the aciculae may be pulverized at the time of molding, and it cannot be necessarily said that the particles have sufficient strength. Since the obtained molded articles deform in subsequent firing, there are problems such as requiring reprocessing. Since hydrothermal synthesis is performed using pressure-resistant reaction containers inside of which can be at a high temperature and a high pressure, such as autoclaves, under severe conditions of a high temperature of 120° C. or more and a pressurized condition in the above-mentioned methods, there is a problem that the device cost and the energy cost are high and productivity is inferior. Therefore, a manufacturing method that can manufacture a material having much better strength, which can also be used as alternative materials to living hard tissue and ivory alternative materials as mentioned above, under milder conditions is desired.

The present disclosure has been completed in view of the problems that conventional technologies have, and an object thereof is to provide a method of manufacturing a composite material molded article in which a composite material molded article having excellent strength containing acicular hydroxyapatite, which can also be used as a material replacing living hard tissue such as artificial bone or artificial teeth or an alternative material to ivory that is difficultly obtained, can be obtained, even when the method is performed under the synthetic conditions at a relatively low temperature (for example, 100° C. or less). Another object of the present disclosure is also to provide a composite material molded article having excellent strength that can also be used, without firing, as the above-mentioned alternative materials to living hard tissue and ivory alternative materials.

### Solution to Problem

To achieve the objects, a method of manufacturing a composite material molded article containing acicular hydroxyapatite according to one aspect of the present disclosure comprises: a preparation step of mixing at least a calcium phosphate compound including α-tricalcium phosphate, a calcium compound containing no phosphorus, cellulose nanofibers, and an aqueous solvent consisting of water and/or a hydrophilic solvent to obtain a mixture; a molding step of forming a molded article using the mixture; a drying step of drying the molded article; and a synthesis step of subjecting the molded article after drying to synthesis treatment.

According to the manufacturing method, a composite material molded article having high strength can be obtained by incorporating the cellulose nanofibers into the mixture forming the composite material molded article without requiring the addition of an organic binder and the like or the firing of the molded article. Here, when a large amount of organic binders such as casein and carboxymethyl cellulose are used, they may coat the surface of α-tricalcium phosphate powder, and there is a problem that acicular hydroxyapatite tends to be hardly produced even when the synthesis treatment is performed. Meanwhile, when cellulose nanofibers are used, acicular hydroxyapatite is produced efficiently without coating the surface of α-tricalcium phosphate powder, acicular hydroxyapatite and the cellulose nanofibers become intertwined, and the strength of the obtained composite material molded article can be improved by leaps and bounds. Since the network of the cellulose nanofibers is formed among particles of α-tricalcium phosphate by strong hydrogen bonds of the cellulose nanofibers by molding and drying the mixture in the manufacturing method, a composite material molded article having very high strength can be obtained, for example, through subsequent synthesis under the condition of saturated water vapor. Additionally, acicular hydroxyapatite can be efficiently produced even when the synthesis treatment is performed at a relatively low temperature (for example, 100° C. or less) using α-tricalcium phosphate as a raw material by the synthesis treatment of the molded article after drying according to the manufacturing method. Acicular hydroxyapatite can be produced without performing the synthesis treatment under severe conditions of a high temperature and a high pressure as in the hydrothermal synthesis, and a composite material molded article in which the strength is improved greatly by integrating this acicular hydroxyapatite and the cellulose nanofibers can be manufactured without firing according to the manufacturing method as mentioned above.

Since artificial bone or a filler consisting of a bioceramic such as tricalcium phosphate or a hydroxyapatite does not generally become strong only by compacting powder similarly to general-purpose ceramics, firing is required. Meanwhile, a composite material molded article having excellent strength can be manufactured without firing in a manufacturing method according to one aspect of the present disclosure. For example, the bending strength of cortical bone (compact bone) is around 50 to 150 MPa, and a composite material molded article having strength that is close to the cortical bone can be obtained according to the manufacturing method according to one aspect of the present disclosure. According to one aspect of the present disclosure, since a firing step is not required, a method of manufacturing a composite material molded article that is also environmentally friendly can be provided.

In one embodiment, the calcium compound may be added in the preparation step to adjust to the Ca/P ratio of hydroxyapatite after synthesis so that the Ca/P ratio (atomic ratio) of the mixture is more than 1.50 and 1.80 or less. Hydroxyapatite to be used as a material of artificial bone or artificial teeth is represented by Ca₁₀(PO₄)₆(OH)₂, and its Ca/P ratio is 1.67. Meanwhile, since the Ca/P ratio of α-tricalcium phosphate is 1.5, the Ca/P ratio may be brought close to 1.67 by adding a calcium compound such as calcium hydroxide. The Ca/P ratio of the obtained composite material molded article can be brought close to 1.67 by adjusting the amount of the calcium compound added so that the Ca/P ratio of the mixture is more than 1.50 and 1.80 or less, and it becomes useful as a biomaterial.

In one embodiment, 10 to 40 parts by mass of the cellulose nanofibers may be added with respect to 100 parts by mass of the calcium phosphate compound in the preparation step. A composite material molded article having more sufficient strength can be obtained by setting the amount of the cellulose nanofibers added at 10 parts by mass or more, and a composite material molded article in which the organic content is moderately low and which is more suitable for a biomaterial can be obtained by setting the amount thereof added at 40 parts by mass or less.

In one embodiment, the manufacturing method may comprise a removal step of removing part or all of the aqueous solvent from the mixture, before the molding step. A reduction in molding time and the formation of the molded article are facilitated by removing a certain level or all of the aqueous solvent from the mixture.

In one embodiment, the molded article may be formed by removing part or all of the aqueous solvent while the mixture is press-molded in the molding step. According to the method, efficiency of operations can be increased, and the formation of the molded article is facilitated. The cellulose nanofibers easily form a firm network by hydrogen bonds at the time of the molding and subsequent drying, and a composite material molded article having higher strength can be obtained by performing molding with the aqueous solvent remaining in the mixture at a certain level, and removing part or all of the aqueous solvent while press-molding.

In one embodiment, the molded article after drying may be subjected to the synthesis treatment at a temperature of 60 to 120° C. or a temperature of 80 to 100° C. in the synthesis step. Since the cellulose nanofibers are incorporated into the mixture, it is preferable to perform the synthesis treatment under a mild temperature condition of 60 to 120° C. The network formed by the hydrogen bonds of the cellulose nanofibers can be maintained by setting to this temperature condition, and a composite material molded article having higher strength can be obtained. According to the manufacturing method, even when the synthesis treatment is performed under the condition of a relatively low temperature of 60 to 120° C., acicular hydroxyapatite can be produced efficiently, and a composite material molded article in which the strength is improved greatly by integrating acicular hydroxyapatite and the cellulose nanofibers can be obtained.

A composite material molded article according to another aspect of the present disclosure comprises acicular hydroxyapatite and cellulose nanofibers. The composite material molded article can obtain excellent strength by containing acicular hydroxyapatite and the cellulose nanofibers.

In one embodiment, in the composite material molded article, a Ca/P ratio may be more than 1.50 and 1.80 or less. The composite material molded article becomes useful as a biomaterial since the Ca/P ratio is near 1.67.

In one embodiment, the composite material molded article may have a structure in which the cellulose nanofibers are hydrogen-bonded to each other. Since cellulose nanofibers are hydrogen-bonded to each other, a film network of cellulose nanofibers is formed, this network and acicular hydroxyapatite become further intertwined, and therefore the composite material molded article can obtain higher strength.

### Advantageous Effects of Invention

According to one aspect and embodiment of the present disclosure, a method of manufacturing a composite material molded article, in which a composite material molded article containing acicular hydroxyapatite having excellent strength, which can also be used as a material replacing living hard tissue such as artificial bone or artificial teeth or an alternative material to ivory that is difficultly obtained, can be obtained even when the synthesis is performed at a relatively low temperature (for example, 100° C. or less) can be provided. According to another aspect and an embodiment of the present disclosure, a composite material molded article having excellent strength, which can also be used as the above-mentioned alternative materials to living hard tissue and ivory alternative materials without firing, can be provided.

### BRIEF DESCRIPTION OF DRAWINGS

FIG. 1 is a flow chart showing the flow of a method of manufacturing a composite material molded article according to one embodiment of the present disclosure.

FIG. 2 is a schematic diagram showing one embodiment of a press-molding machine used in the molding step.

FIG. 3 is a schematic diagram showing the filtration filter of the press-molding machine.

FIG. 4 is electron microscope photographs of a composite material molded article obtained in Example 1.

FIG. 5 is electron microscope photographs of a composite material molded article obtained in Example 2.

FIG. 6 is electron microscope photographs of a composite material molded article obtained in Comparative Example 1.

FIG. 7 is electron microscope photographs of a composite material molded article obtained in Comparative Example 2.

FIG. 8 is XRD patterns of the molded article of Example 1 before and after treatment.

FIG. 9 is XRD patterns of the molded article of Example 2 before and after treatment.

FIG. 10 is XRD patterns of the molded article of Comparative Example 1 before and after treatment.

FIG. 11 is XRD patterns of the molded article of Comparative Example 2 before and after treatment.

### DESCRIPTION OF EMBODIMENTS

Preferred embodiments of the present disclosure will be described in detail hereinafter with reference to the drawings. The same or corresponding parts are indicated with the same sign in the drawings, and the duplicative description is omitted. Dimensional ratios of the drawings are not limited to ratios shown.

A method of manufacturing a composite material molded article containing acicular hydroxyapatite of the present disclosure is a method comprising: a preparation step of mixing at least a calcium phosphate compound including α-tricalcium phosphate, a calcium compound containing no phosphorus, cellulose nanofibers, and an aqueous solvent consisting of water and/or a hydrophilic solvent to obtain a mixture; a molding step of forming a molded article using the mixture; a drying step of drying the molded article; and a synthesis step of subjecting the molded article after drying to synthesis treatment. The manufacturing method may further comprise a removal step of removing part or all of the aqueous solvent from the mixture, before the molding step and after the preparation step. The manufacturing method may further comprise a second drying step of drying the molded article after synthesis, after the synthesis step.

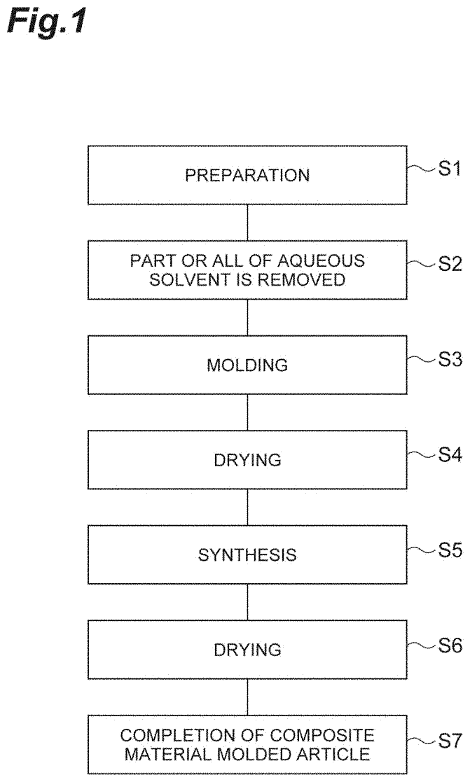

FIG. 1 is a flow chart showing the flow of a method of manufacturing a composite material molded article according to one embodiment of the present disclosure. As shown in FIG. 1, a composite material molded article goes through preparation step S1 of mixing materials to obtain a mixture, removal step S2 of removing part or all of an aqueous solvents contained in the obtained mixture from the mixture, molding step S3 of molding the mixture from which a certain level or all of the aqueous solvent is removed to obtain a molded article, drying step S4 of drying the obtained molded article, synthesis step S5 of subjecting the molded article after drying to the synthesis treatment, and second drying step S6 of drying the molded article after synthesis, and is completed (S7) in the manufacturing method of the present embodiment. The steps will be described in detail hereinafter.

(Preparation Step S1)

In preparation step S1, at least a calcium phosphate compound including α-tricalcium phosphate, a calcium compound such as calcium hydroxide containing no phosphorus, cellulose nanofibers, and an aqueous solvent consisting of water and/or a hydrophilic solvent are mixed to obtain a mixture. As long as the mixing method is a method capable of mixing materials sufficiently, it is not particularly limited. Mixing can be performed by stirring, for example, by using a stirrer, a hand mixer, or an automatic mortar. As long as the mixing method is a method that does not damage the cellulose nanofibers, it is not particularly limited. A mixing method such as a homogenizer that may damage the cellulose nanofibers is not preferable.

α-Tricalcium phosphate is a particulate material that is represented by Ca₃(PO₄)₂, and the Ca/P (atomic ratio) ratio of which is 1.5. α-Tricalcium phosphate has the property of converting into the hydroxyapatite (Ca₁₀(PO₄)₆(OH)₂), that is the main component of bone, gradually in water. Although α-tricalcium phosphate is particulate, acicular hydroxyapatite is produced from the particle surface by synthesizing, for example, under the conditions of 60 to 120° C. and around 6 to 24 hours (in contact with water vapor in an airtight container). In the present disclosure, an acicular shape includes shapes such as a needle shape, a fiber shape, a rod shape and a plate shape.

Tricalcium phosphate has an a type (high temperature stable phase) and a β type (low temperature stable phase). In the present embodiment, it is essential to use a type tricalcium phosphate (α-tricalcium phosphate) as a raw material to produce acicular hydroxyapatite by synthesis. When β type tricalcium phosphate is used as a raw material, it is difficult to convert it into acicular hydroxyapatite even when the synthesis treatment is performed. However, when β-tricalcium phosphate is heated to 1170° C. or more, the crystal structure changes into that of α-tricalcium phosphate. Therefore, β-tricalcium phosphate may be used as a starting material, and a material that is thermally changed into α-tricalcium phosphate by heating at a temperature of 1170° C. or more, preferably 1200 to 1400° C. or more than 1400° C. may be used in the present embodiment. α Type tricalcium phosphate (high temperature stable phase) has a monoclinic system (α-TCP) and a hexagonal system (α′-TCP), and both can be used in the present disclosure. Since α-TCP is excellent in reactivity with water and easily converts into acicular hydroxyapatite, it is more preferable among α-TCP and α′-TCP. Tricalcium phosphate can be used alone or in combination of two or more.

Although the particle size of α-tricalcium phosphate is not particularly limited, it is preferable that the average particle size be 3 to 15 μm, and it is more preferable that the average particle size be 3 to 8 μm from the viewpoint of obtaining sufficient strength of the composite material molded article and the viewpoint of producing acicular hydroxyapatite at a high aspect ratio efficiently by synthesis. The particle size can be measured by laser diffractometry.

A calcium phosphate compound other than α-tricalcium phosphate may be added to the mixture as a calcium phosphate compound. Examples of the other calcium phosphate compound include calcium hydrogen phosphate, calcium hydrogen phosphate dihydrate, tetracalcium phosphate, octacalcium phosphate and calcium metaphosphate. Since the adjustment such as increasing reactivity or reacting slowly is enabled by adding the other calcium phosphate compound, the microstructure of the resultant composite material molded article can be changed, and strength adjustment (improvement or reduction) is enabled. The other calcium phosphate compound can be used alone or in combination of two or more.

When the other calcium phosphate compound is added to the mixture, it is preferable that the amount thereof added be such that the molar ratio of the other calcium phosphate compound to α-tricalcium phosphate (the number of the moles of the other calcium phosphate compounds/the number of the moles of α-tricalcium phosphate) be 0.5 or less, and it is more preferable that the amount thereof added be such that the ratio be 0.25 or less. When the molar ratio is 0.5 or less, a sufficient proportion of α-tricalcium phosphate exists, and therefore a composite material molded article having high strength containing acicular hydroxyapatite is easily obtained.

A calcium compound containing no phosphorus (compound containing no phosphorus atom but containing a calcium atom in a molecule) is used to adjust the Ca/P ratio of hydroxyapatite after synthesis. The calcium compound means a calcium compound other than a compound containing phosphorus such as a calcium phosphate compound. Examples of the calcium compound include calcium hydroxide, calcium chloride, calcium nitrate, calcium nitrate hydrate, calcium sulfate, calcium carbonate, calcium carbonate hydrate, and organic acid calcium (calcium acetate, calcium lactate and the like). Among these, calcium hydroxide (Ca(OH)₂) is particularly preferable. A general calcium compound can be used without particular limitation. The calcium compound can be used alone or in combination of two or more. As mentioned above, a calcium phosphate compound other than α-tricalcium phosphate can be incorporated into the mixture.

It is preferable that the amount of the calcium compound containing no phosphorus added in the mixture be an amount in which the Ca/P ratio of the mixture be more than 1.50 and 1.80 or less, it is more preferable that it be an amount in which the Ca/P ratio be 1.66 to 1.70, and it is particularly preferable that it be an amount in which the Ca/P ratio be 1.67. The Ca/P ratio of the obtained composite material molded article approaches 1.67 by adjusting the amount of the calcium compound added as mentioned above, and it becomes useful as a biomaterial.

The cellulose nanofibers are a biomass material obtained by highly nanosizing (micronizing) wood fibers (pulp) obtained from wood to a nano order, that is one several-hundredth of one micron or smaller. Since the cellulose nanofibers are derived from vegetable fibers, they are characterized by having a small environmental load as to production and disposal and being light. The cellulose nanofibers have excellent characteristics of a high elastic modulus and small expansion and contraction accompanying temperature change. A composite material molded article having very high strength can be obtained by adding these cellulose nanofibers in the mixture, subjecting the resultant to molding, drying and synthesis, and integrating the resultant with acicular hydroxyapatite. The cellulose nanofibers can be used alone or in combination of two or more.

It is preferable that the amount of the cellulose nanofibers added in the mixture be 5 to 40 parts by mass, it is more preferable that it be 10 to 30 parts by mass, it is further preferable that it be 15 to 30 parts by mass, and it is particularly preferable that it be 20 to 30 parts by mass with respect to 100 parts by mass of the calcium phosphate compound (total amount of α-tricalcium phosphate and the other calcium phosphate compound added if needed). When the amount thereof added is 5 parts by mass or more, a composite material molded article having more sufficient strength tends to be able to be obtained, and when it is 40 parts by mass or less, a composite material molded article in which the organic content is moderately low and which is more suitable for a biomaterial tends to be able to be obtained.

As the aqueous solvent, water, a hydrophilic solvent, or a mixed solvent thereof can be used. Since the cellulose nanofibers have excellent dispersibility to water, it is preferable to use water as an aqueous solvent.

As the water, distilled water, ion-exchanged water, pure water, ultrapure water, tap water or the like can be used. Among these, distilled water, ion-exchanged water, pure water and ultrapure water are preferable.

As long as the hydrophilic solvent is a solvent compatible with water, there is no particular problem, but it is environmentally preferable to use 99.5% ethanol, ethanol for industry, and ethanol for disinfection.

Since the amount of the aqueous solvent in the mixture added varies depending on the type of the solvent and the concentration and type of the cellulose nanofibers, it cannot be unconditionally specified; however, it is preferable that an amount in which the cellulose nanofibers can be dispersed adequately be 500 to 1000 parts by mass with respect to 100 parts by mass of the calcium phosphate compound.

Materials other than the above may be added to the mixture. For example, phosphoric acid may be added to the mixture. Since the adjustment such as increasing reactivity or reacting slowly can be achieved by adding phosphoric acid, the microstructure of the resultant composite material molded article can be changed, and strength adjustment (improvement or reduction) is enabled.

A polylactic acid emulsion (biodegradable resin) may be added to the mixture for further improving strength.

(Removal Step S2)

In removal step S2, part or all of the aqueous solvent contained in the mixture produced in preparation step S1 is removed from the mixture. Examples of the method of removing the aqueous solvent include methods such as drying, filtration and centrifugal separation. Examples of the drying method include drying at normal temperature and normal pressure, drying by warming, vacuum drying and freeze drying. The mixture may be turned into mixed powder containing no aqueous solvent by removing the aqueous solvent by these methods. When the raw materials are mixed by an automatic mortar in preparation step S1, the aqueous solvent may be removed by continuing stirring by the automatic mortar at normal temperature and normal pressure sequentially until the mixture becomes powdered. It is preferable to perform the removal step at a temperature of 40° C. or less, and it is more preferable to perform it at a temperature of normal temperature (25° C.) or less from the viewpoint of suppressing the conversion of α-tricalcium phosphate into hydroxyapatite.

The content of the aqueous solvent remaining in the mixture after removal step S2 is not particularly limited and may be any as long as the molded article can be manufactured by the molding method in molding step S3, but it is preferable that it be set in the range in which the molding of a molded article be facilitated. When part or all of the aqueous solvent is removed to form the molded article while the mixture is press-molded, the content of the aqueous solvent remaining in the mixture may be 50 to 80% by mass, and may be 60 to 70% by mass on the basis of the total amount of the mixture. When all the aqueous solvent is removed from the mixture in removal step S2, the aqueous solvent does not remain, and therefore molding by press-molding or the like can be performed without being accompanied with the removal of the aqueous solvent in the subsequent molding step S3.

(Molding Step S3)

The mixture used in molding step S3 may be either of a mixture from which the aqueous solvent was removed by removal step S2 or a mixture in which a certain level of the aqueous solvent remains. These mixtures (raw material mixtures) are molded in molding step S3 to obtain a molded article. It is preferable to perform molding by press-molding. Press-molding can be performed by pressurizing the mixed powder from which the aqueous solvent was removed. Even when it contains the aqueous solvent, it may be press-molded by pressurizing while the aqueous solvent is volatilized by heating it to around 100° C. It may be dried after it is molded at normal temperature. Additionally, molding may be performed while pressure is reduced.

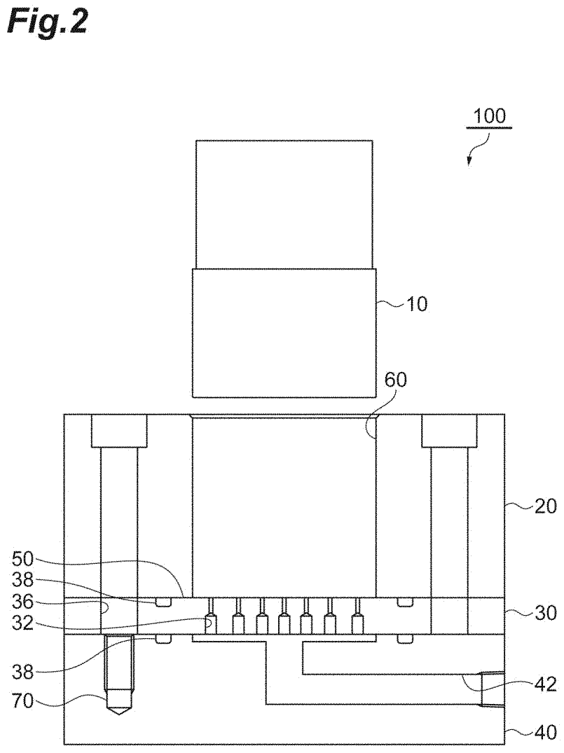

When molding step S3 is performed by press-molding, it is preferable to remove part or all of the aqueous solvent to form a molded article while the mixture is press-molded. Examples of this method include a method of press-molding while the aqueous solvent is removed using a press-molding machine having a structure, for example, as shown in FIG. 2. A press-molding machine 100 shown in FIG. 2 comprises a punch 10, a die (molding die) 20, a filtration filter 30 and a base 40, and the die 20, the filtration filter 30 and the base 40 are stacked and fixed with bolts 70 through bolt holes 36. A membrane filter 50 is disposed between the die 20 and the filtration filter 30 in the sandwiched state. Molding is perforated by feeding the mixture to a cavity 60 of the die 20 and pressurizing it with the punch 10 while pressure in the cavity 60 is reduced. At this time, the aqueous solvent contained in the mixture is extracted with the membrane filter 50 and the filtration filter 30 disposed at the bottom of the cavity 60, and is discharged through holes 32 provided in the filtration filter 30 and a drainage channel 42 provided in the base 40.

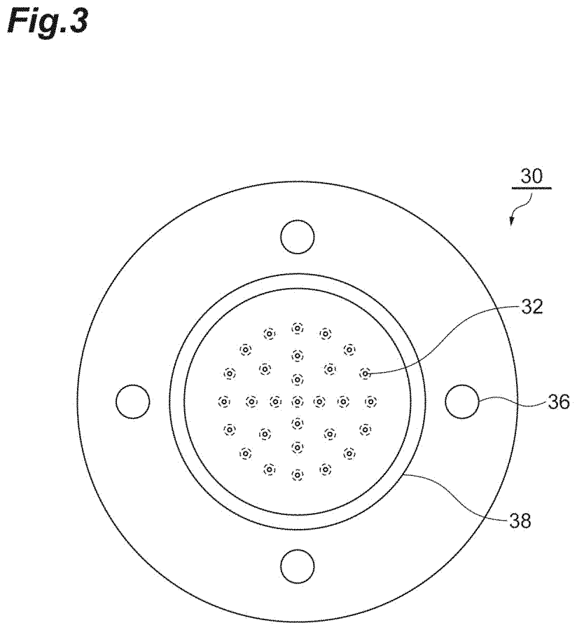

FIG. 3 is a schematic diagram showing the filtration filter 30 of the press-molding machine 100. FIG. 3 is a diagram in which the filtration filter 30 is viewed from the die 20 side in FIG. 2. As shown in FIG. 3, many holes 32 for extracting the aqueous solvent from the mixture are provided in the filtration filter 30. The diameter of the holes 32 is, for example, a diameter of around 1 mm from the surface to a fixed depth, and is a diameter of around 3 mm from there to the opposite surface. The diameter of the holes 32 can be adjusted properly. An O-ring 38 is disposed at the periphery of the many holes 32. A molded article can be formed by press-molding the mixture by using the press-molding machine 100 provided with the filtration filter 30 while part or all of the aqueous solvent is removed.

(Drying Step S4)

In drying step S4, the molded article produced in molding step S3 is unmolded and dried in a drier at a temperature of normal temperature to 50° C., preferably at a temperature of 30 to 50° C., and more preferably at a temperature of 40 to 50° C. for 24 to 48 hours.

The content of the aqueous solvent remaining in the molded article after drying step S4 may be 0.5% by mass or less (0 to 0.5% by mass), and may be 0.1% by mass or less (0 to 0.1% by mass) on the basis of the total amount of the molded article. Since the content of the aqueous solvent remaining is in the range, the network by the hydrogen bonds of the cellulose nanofibers can be formed sufficiently in drying step S4 and synthesis step S5, and acicular hydroxyapatite can be efficiently produced in synthesis step S5.

(Synthesis Step S5)

In synthesis step S5, synthesis is performed by the treatment of bringing the molded article dried in drying step S4 in contact with water vapor in an airtight container preferably at a temperature of 120° C. or less, more preferably at a temperature of 60 to 120° C., and further preferably at a temperature of 80 to 100° C. for 6 to 120 hours. α-Tricalcium phosphate can be converted into acicular hydroxyapatite by performing synthesis under the above-mentioned conditions. In synthesis step S5, a large-scale device such as an autoclave is not required, but a container that can be closed airtightly can be used without particular limitation.

(Second Drying Step S6)

In second drying step S6, the molded article after synthesis is dried in the drier at a temperature of normal temperature to 50° C., and preferably at a temperature of 30 to 50° C. for 6 hours or more. The aqueous solvent remaining in the molded article and water adhering to the molded article at the time of synthesis are removed by this.

According to the manufacturing method of the present embodiment, the composite material molded article in which the strength is improved greatly by integrating acicular hydroxyapatite and the cellulose nanofibers can be manufactured through the above-mentioned steps. The manufacturing method of the present embodiment can be a manufacturing method not having a firing step. The composite material molded article having excellent strength can be obtained without performing firing, for example, at a temperature of more than 120° C. according to the manufacturing method of the present embodiment.

Next, one embodiment of the composite material molded article of the present disclosure will be described. The composite material molded article of the present embodiment contains acicular hydroxyapatite and cellulose nanofibers.

It is preferable that the Ca/P ratio of the composite material molded article be more than 1.50 and 1.80 or less, it is more preferable that it be 1.66 to 1.68, and it is particularly preferable that it be 1.67. The composite material molded article becomes useful as a biomaterial since it has the above Ca/P ratio. The Ca/P ratio of the composite material molded article can be measured with an ICP emission spectrometer (quantitative analysis), a fluorescence X-ray diffractometer, an energy dispersive X-ray microanalyzer or the like.

It is preferable that the composite material molded article have a structure in which cellulose nanofibers be hydrogen-bonded to each other. It is preferable that the composite material molded article have a structure in which the network formed by the hydrogen bonds of the cellulose nanofibers and acicular hydroxyapatite be intertwined and integrated. Such a structure can be confirmed, for example, by electron microscope observation. The composite material molded article can obtain excellent strength since it has such a structure. The composite material molded article having such a structure can be manufactured by the above-mentioned method of manufacturing a composite material molded article.

Although preferred embodiments of the method of manufacturing a composite material molded article and the composite material molded article of the present disclosure were described in detail above, the present disclosure is not limited to the above-mentioned embodiments, and various variations and modifications are possible in the scope of the present disclosure described in Claims.

The composite material molded article manufactured by the manufacturing method of the present disclosure and the composite material molded article of the present disclosure can be suitably used as a material replacing living hard tissue such as artificial bone or artificial teeth or an alternative material to ivory that is difficultly obtained. The shape of the composite material molded article is not particularly limited, but the composite material molded article can be processed into a desired shape depending on a specific use after it is manufactured. When the composite material molded article is manufactured by the manufacturing method of the present disclosure, it may be molded into a desired shape depending on a specific use beforehand in the molding step.

### EXAMPLES

Although the present disclosure will be more specifically described on the basis of Examples and Comparative Examples hereinafter, the present disclosure is not limited to the following Examples.

### Example 1

After 20 parts by mass (solid content) of cellulose nanofibers were sufficiently dispersed in 900 parts by mass of distilled water, 90.12 parts by mass of α-tricalcium phosphate, 9.88 parts by mass of calcium hydrogen phosphate (molar ratio of α-tricalcium phosphate to calcium hydrogen phosphate=4:1), and a predetermined amount of calcium hydroxide were added thereto, and stirring and mixing were performed with a hand mixer for 5 minutes to prepare a mixture (preparation step). Here, the amount of calcium hydroxide blended was such an amount that the Ca/P ratio of the obtained mixture was 1.67.

The obtained mixture was dehydrated and filtrated through a membrane filter for around 3 hours, and the content of water remaining in the mixture was adjusted to 60 to 70% by mass (removal step). The mixture from which a certain level of water was removed in the removal step was fed to the cavity of the press-molding machine shown in FIG. 2, and pressure molding was performed slowly while the pressure was reduced in the cavity. A membrane filter and a filtration filter provided with holes of around 1 mm were disposed at the bottom of the cavity of a press-molding machine, and the solvent was extracted to form a molded article while pressure molding was performed (molding step).

The molded article after unmolding was dried in a drier at 40 to 50° C. for 72 hours (drying step). Subsequently, the molded article after drying was synthesized under the conditions of 80 to 100° C. and 24 hours (synthesis step). Synthesis was performed by bringing the molded article in contact with water vapor in a glass airtight container. A composite material molded article containing acicular hydroxyapatite and the cellulose nanofibers was obtained by drying the molded article after synthesis at normal temperature to 50° C. for 72 hours (second drying step).

### Example 2

After 20 parts by mass (solid content) of the cellulose nanofibers were sufficiently dispersed in 900 parts by mass of distilled water, 100 parts by mass of α-tricalcium phosphate and a predetermined amount of calcium hydroxide were added thereto, and stirring and mixing were performed with the hand mixer for 5 minutes to prepare a mixture (preparation step). Here, the amount of calcium hydroxide blended was such an amount that the Ca/P ratio of the obtained mixture was 1.67. The removal step, the molding step, the drying step, the synthesis step and the second drying step were performed in the same way as in Example 1 except that the mixture obtained in the above preparation step was used to obtain a composite material molded article containing acicular hydroxyapatite and the cellulose nanofibers.

### Comparative Example 1

After 20 parts by mass (solid content) of the cellulose nanofibers were sufficiently dispersed in 900 parts by mass of distilled water, 100 parts by mass of calcium hydrogen phosphate and a predetermined amount of calcium hydroxide were added thereto, and stirring and mixing were performed with the hand mixer for 5 minutes to prepare a mixture (preparation step). Here, the amount of calcium hydroxide blended was such an amount that the Ca/P ratio of the obtained mixture was 1.67. The removal step, the molding step, the drying step, the synthesis step and the second drying step were performed in the same way as in Example 1 except that the mixture obtained in the above preparation step was used to obtain a composite material molded article.

### Comparative Example 2

100 parts by mass of hydroxyapatite, 20 parts by mass (solid content) of the cellulose nanofibers and 900 parts by mass of distilled water were stirred and mixed with the hand mixer for 5 minutes to prepare a mixture (preparation step). The removal step, the molding step, the drying step, the synthesis step and the second drying step were performed in the same way as in Example 1 except that the mixture obtained in the above preparation step was used to obtain a composite material molded article.

The materials used for preparing the mixtures and the amounts thereof blended in Examples and Comparative Examples were shown together in table 1.

The unit of the amounts thereof blended shown in table 1 is parts by mass, and the amounts of the materials other than the solvent blended show the amounts of solid contents blended. Details of the materials in table 1 are as follows.

(Particulate Bone Material)

α-Tricalcium phosphate (α-TCP): Ca₃(PO₄)₂, manufactured by TAIHEI CHEMICAL INDUSTRIAL CO., LTD., and Ca/P ratio=1.5

Calcium hydrogen phosphate (dicalcium phosphate anhydride, DCPA): CaHPO₄, manufactured by TAIHEI CHEMICAL INDUSTRIAL CO., LTD., and Ca/P ratio=1

Hydroxyapatite (HAp): Ca₁₀(PO₄)₆(OH)₂, manufactured by TAIHEI CHEMICAL INDUSTRIAL CO., LTD., and Ca/P ratio=1.67

Calcium hydroxide: Ca(OH)₂, manufactured by Wako Pure Chemical Industries, Ltd.

### (Filler)

Cellulose nanofiber: manufactured by SUGINO MACHINE LIMITED, trade name “BiNFi-s”

<Analysis of Composite Material Molded Article>

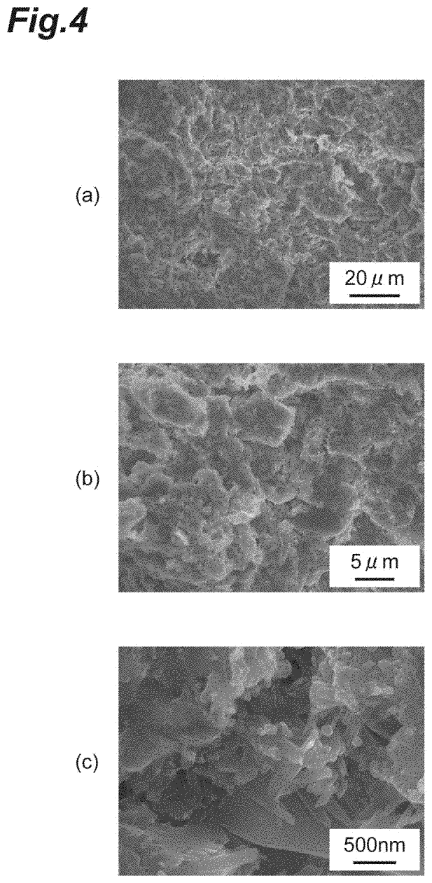

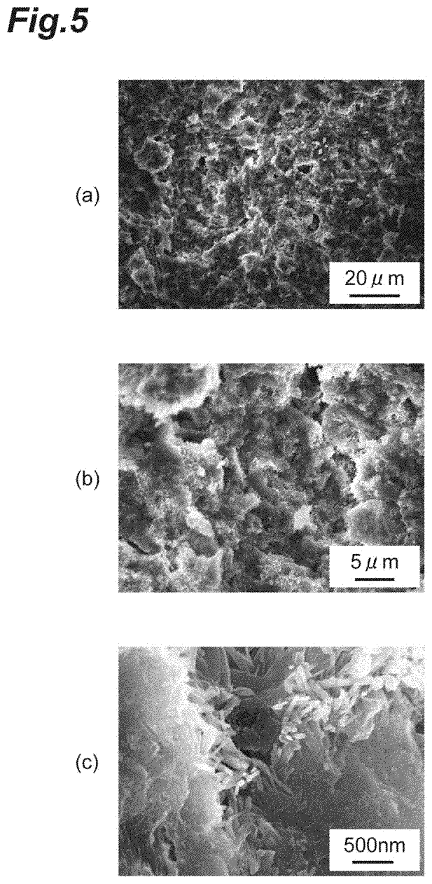

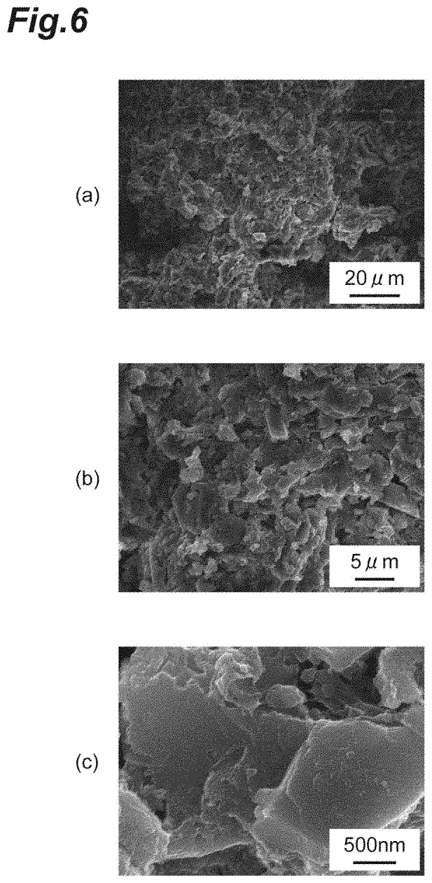

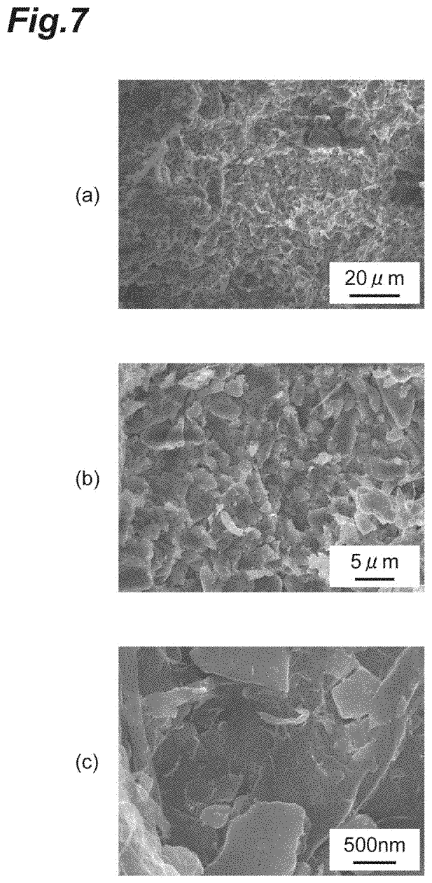

The composite material molded articles obtained in Examples and Comparative Examples were observed by using a scanning electron microscope (manufactured by JEOL Ltd., JSM-7500F). Scanning electron microscope (SEM) photographs (magnification: 1000, 3000 and 30000 times) of the sections (insides) of the composite material molded articles obtained by Examples 1 to 2 and Comparative Examples 1 to 2 are shown in FIGS. 4 to 7. (a), (b) and (c) in FIGS. 4 to 7 are SEM photographs taken at different magnifications, the (a) is at a magnification of 1000 times, (b) is at a magnification of 3000 times, and (c) is at a magnification of 30000 times. In FIG. 4 (Example 1) and FIG. 5 (Example 2), it can be confirmed that acicular deposits (around 50 nm in diameter and 500 nm in length) are intertwined and deposited in the composite material molded articles obtained in Examples 1 and 2. Meanwhile, in FIG. 6 (Comparative Example 1) and FIG. 7 (Comparative Example 2), acicular deposits could not be confirmed in the composite material molded articles obtained in Comparative Examples 1 and 2.

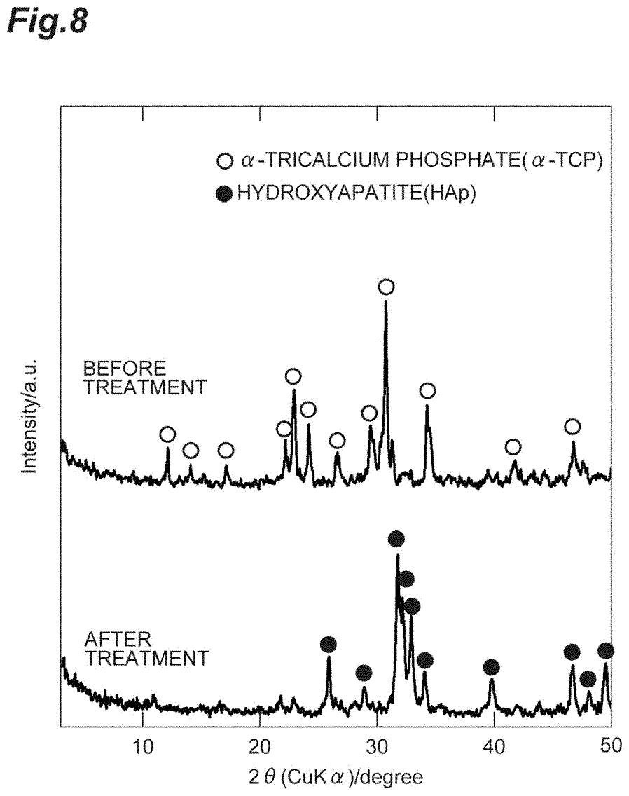

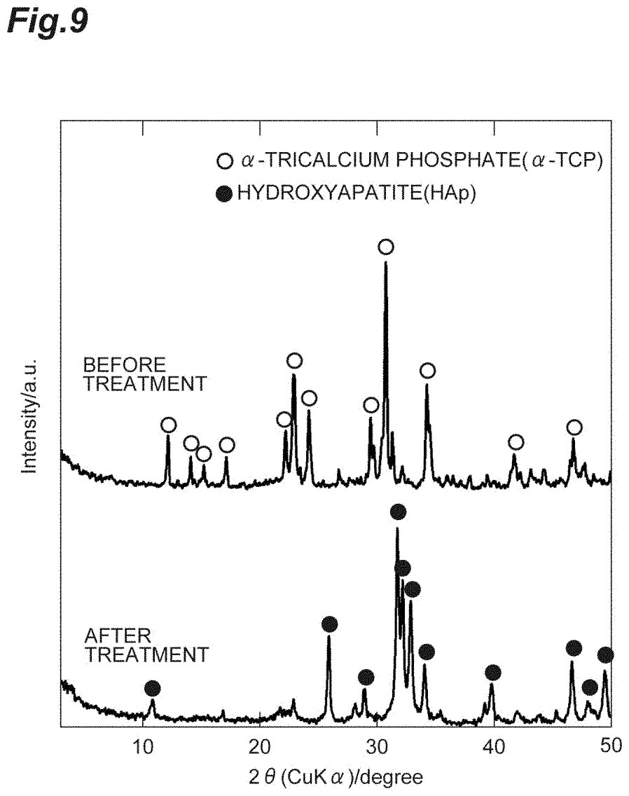

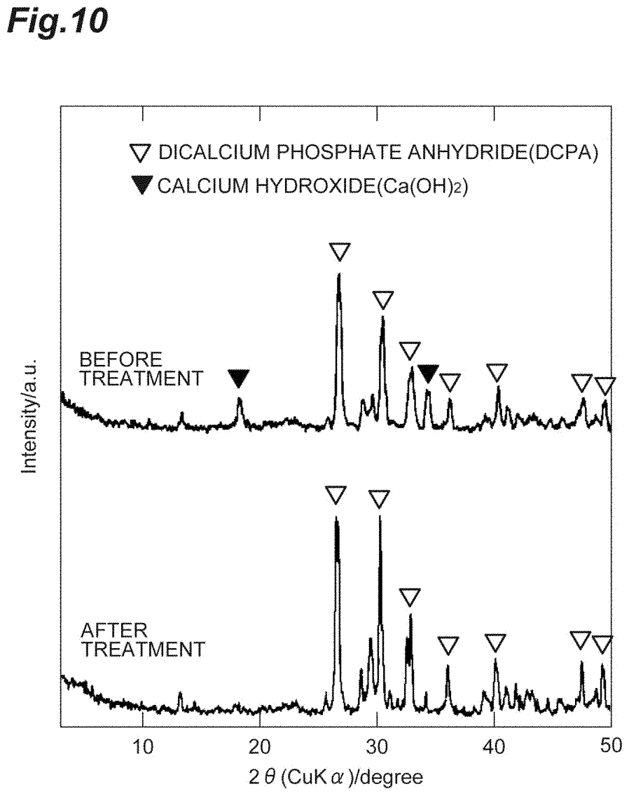

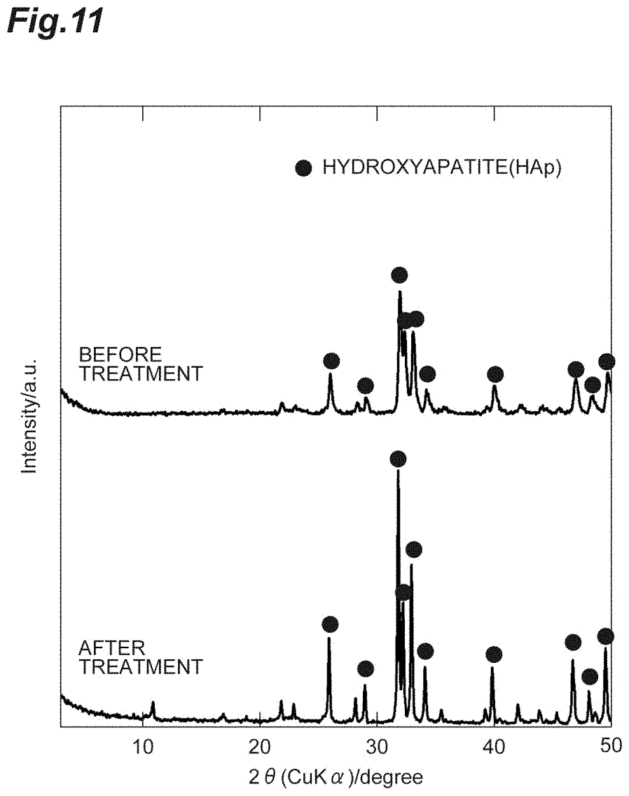

The powder X-ray diffraction (XRD) patterns of the mixed powders of the raw materials before synthesis treatment and the crystal phases of the composite material molded articles obtained by synthesis treatment in Examples and Comparative Examples were measured in the range of 2θ=3° to 50° by using an X-ray diffraction device (manufactured by Rigaku Corporation, trade name “R1NT2100”, radiation source: Cu Kα line). The powder XRD patterns of the mixed powders of the raw materials before synthesis treatment (before treatment) and the composite material molded articles after synthesis treatment (after treatment) of Examples 1 to 2 and Comparative Examples 1 to 2 are shown in FIGS. 8 to 11. As shown in FIG. 8 (Example 1) and FIG. 9 (Example 2), the peaks of the α-tricalcium phosphate (α-TCP) derived from the raw materials disappeared after the synthesis treatment, and only peaks that belonged to hydroxyapatite (HAp) were observed in Examples 1 and 2. From this result and the results of the SEM photographs, it was confirmed that the acicular deposits in the composite material molded articles of Examples 1 and 2 were hydroxyapatite. Meanwhile, as shown in FIG. 10, the peaks of calcium hydrogen phosphate (DCPA) derived from the raw materials did not disappear even after synthesis treatment, and peaks that belonged to hydroxyapatite (HAp) were not observed in Comparative Example 1. From these results, it was confirmed that hydroxyapatite was not produced in the composite material molded article of Comparative Example 1. As shown in FIG. 11, the peaks of the raw material mixture that belonged to hydroxyapatite (HAp) derived from the raw materials were observed at substantially the same position as those before and after synthesis treatment in Comparative Example 2. From this result and the results of the SEM photographs, it was confirmed that hydroxyapatite that is a raw material remained the same without changing into aciculae in the composite material molded article of Comparative Example 2.

<Measurement of Bending Strength>

The composite material molded articles obtained in the Examples and Comparative examples were processed into platy specimens of 8±1 mm×40±1 mm×2.2±0.5 mm in thickness. The three-point bending tests of these specimens were performed by using a strength test (manufactured by INSTRON, trade name “Instron 5566”). Measurement conditions were set as distance between fulcrums: 15±2 mm, measurement speed (moving speed of head): 1.00 mm/min, and measurement temperature: room temperature (10 to 35° C.). The average value of five specimens was found and defined as a measurement result. The results are shown in Table 1.

TABLE 1

| | | | | Comparative | Comparative |
|---|---|---|---|---|---|
| | | Example 1 | Example 2 | Example 1 | Example 2 |
| Composition of mixture | α-Tricalcium phosphate | 90.12 | 100 | — | — |
| (part by mass) | Calcium hydrogen | 9.88 | — | 100 | — |
| | phosphate (dicalcium | | | | |
| | phosphate anhydride) | | | | |
| | Hydroxyapatite | — | — | — | 100 |
| | Calcium hydroxide | Amount wherein | Amount wherein | Amount wherein | — |
| | | Ca/P ratio is 1.67 | Ca/P ratio is 1.67 | Ca/P ratio is 1.67 | |
| | Cellulose nanofiber | 20 | 20 | 20 | 20 |
| | Distilled water | 900 | 900 | 900 | 900 |

| | | | | |
|---|---|---|---|---|
| Ca/P ratio of mixture | 1.67 | 1.67 | 1.67 | 1.67 |
| Treatment temperature (° C.) | 80-100 | 80-100 | 80-100 | 80-100 |
| Whether acicular HAp exists in | Exist | Exist | Not exist | Not exist |
| composite material molded article | | | | |
| or not | | | | |
| Bending strength of composite | 46 | 40 | 16 | 16 |
| material molded article (MPa) | | | | |

### Example 3

After 20 parts by mass (solid content) of the cellulose nanofibers were sufficiently dispersed in 900 parts by mass of distilled water, 90.12 parts by mass of α-tricalcium phosphate, 9.88 parts by mass of calcium hydrogen phosphate (molar ratio of α-tricalcium phosphate to calcium hydrogen phosphate=4:1), and a predetermined amount of calcium hydroxide were added thereto, and stirring and mixing were performed with an automatic mortar for 30 minutes or more to prepare a mixture (preparation step). Here, the amount of calcium hydroxide blended was such an amount that the Ca/P ratio of the obtained mixture was 1.67.

The aqueous solvent was removed from the mixture by continuing stirring at normal temperature and normal pressure with the automatic mortar until it becomes powdered (removal step). The obtained mixture was preliminary molded with a die, and it was molded into a molded article by cold isostatic pressing (molding step). The molded article after unmolding was dried by maintaining it at normal temperature and normal pressure (drying step). Subsequently, the molded article after drying was synthesized under the conditions of 80 to 100° C. and 24 hours (synthesis step). Synthesis was performed by bringing the molded article in contact with water vapor in a glass airtight container. A composite material molded article containing acicular hydroxyapatite and the cellulose nanofibers was obtained by drying the molded article after synthesis at normal temperature to 50° C. for 72 hours (second drying step).

When the composite material molded article obtained by the method was observed through the scanning electron microscope and analyzed with the X-ray diffraction device, it was confirmed that acicular hydroxyapatite deposited in the composite material molded article. The bending strength of the obtained composite material molded article was substantially the same as that of the composite material molded article of Example 1.

As is clearly from the above results, it was confirmed that the composite material molded article containing acicular hydroxyapatite and the cellulose nanofibers and having excellent strength was obtained even though synthesis was performed at a low temperature of 100° C. or less by the manufacturing method of Example.

### INDUSTRIAL APPLICABILITY

According to a method of manufacturing a composite material molded article of the present disclosure, a composite material molded article containing acicular hydroxyapatite and having excellent strength can be obtained even though synthesis was performed at a relatively low temperature (for example, 100° C. or less). The composite material molded article obtained by the present disclosure is useful as a material replacing living hard tissue such as artificial bone, artificial teeth or artificial dental roots or an alternative material to ivory that is difficultly obtained. The composite material molded article obtained by the present disclosure can also be used as a material that adsorbs and removes proteins and toxic substances and applied to the environmental field.

### REFERENCE SIGNS LIST

10: punch, 20: die, 30: filtration filter, 32: hole, 40: base, 42: drainage channel, 50: membrane filter, 60: cavity, 100: press-molding machine.

1. A method of manufacturing a composite material molded article containing acicular hydroxyapatite, comprising:
a preparation step of mixing at least a calcium phosphate compound including α-tricalcium phosphate, a calcium compound containing no phosphorus, cellulose nanofibers, and an aqueous solvent consisting of water and/or a hydrophilic solvent to obtain a mixture;
a molding step of forming a molded article using the mixture;
a drying step of drying the molded article; and
a synthesis step of subjecting the molded article after drying to synthesis treatment.

2. The method of manufacturing a composite material molded article containing acicular hydroxyapatite according to claim 1, wherein in the preparation step, the calcium compound is added so that a Ca/P ratio of the mixture is more than 1.50 and 1.80 or less.

3. The method of manufacturing a composite material molded article containing acicular hydroxyapatite according to claim 1, wherein in the preparation step, the cellulose nanofibers are added at 10 to 40 parts by mass with respect to 100 parts by mass of the calcium phosphate compound.

4. The method of manufacturing a composite material molded article containing acicular hydroxyapatite according to claim 1, comprising, before the molding step:
a removal step of removing part or all of the aqueous solvent from the mixture.

5. The method of manufacturing a composite material molded article containing acicular hydroxyapatite according to claim 1, wherein in the molding step, part or all of the aqueous solvent was removed while the mixture is press-molded to form the molded article.

6. The method of manufacturing a composite material molded article containing acicular hydroxyapatite according to claim 1, wherein in the synthesis step, the molded article after drying is subjected to the synthesis treatment at a temperature of 60 to 120° C.

7. A composite material molded article, comprising: acicular hydroxyapatite and cellulose nanofibers.

8. The composite material molded article according to claim 7, wherein a Ca/P ratio is more than 1.50 and 1.80 or less.

9. The composite material molded article according to claim 7, having a structure in which the cellulose nanofibers are hydrogen-bonded to each other.

10. The method of manufacturing a composite material molded article containing acicular hydroxyapatite according to claim 2, wherein in the preparation step, the cellulose nanofibers are added at 10 to 40 parts by mass with respect to 100 parts by mass of the calcium phosphate compound.

11. The method of manufacturing a composite material molded article containing acicular hydroxyapatite according to claim 2, comprising, before the molding step:
a removal step of removing part or all of the aqueous solvent from the mixture.

12. The method of manufacturing a composite material molded article containing acicular hydroxyapatite according to claim 3, comprising, before the molding step:
a removal step of removing part or all of the aqueous solvent from the mixture.

13. The method of manufacturing a composite material molded article containing acicular hydroxyapatite according to claim 2, wherein in the molding step, part or all of the aqueous solvent was removed while the mixture is press-molded to form the molded article.

14. The method of manufacturing a composite material molded article containing acicular hydroxyapatite according to claim 3, wherein in the molding step, part or all of the aqueous solvent was removed while the mixture is press-molded to form the molded article.

15. The method of manufacturing a composite material molded article containing acicular hydroxyapatite according to claim 4, wherein in the molding step, part or all of the aqueous solvent was removed while the mixture is press-molded to form the molded article.

16. The method of manufacturing a composite material molded article containing acicular hydroxyapatite according to claim 2, wherein in the synthesis step, the molded article after drying is subjected to the synthesis treatment at a temperature of 60 to 120° C.

17. The method of manufacturing a composite material molded article containing acicular hydroxyapatite according to claim 3, wherein in the synthesis step, the molded article after drying is subjected to the synthesis treatment at a temperature of 60 to 120° C.

18. The method of manufacturing a composite material molded article containing acicular hydroxyapatite according to claim 4, wherein in the synthesis step, the molded article after drying is subjected to the synthesis treatment at a temperature of 60 to 120° C.

19. The method of manufacturing a composite material molded article containing acicular hydroxyapatite according to claim 5, wherein in the synthesis step, the molded article after drying is subjected to the synthesis treatment at a temperature of 60 to 120° C.

20. The composite material molded article according to claim 8, having a structure in which the cellulose nanofibers are hydrogen-bonded to each other.
